(19) United States
(12) Patent Application Publication
(10) Pub. No.: US 2008/0171941 A1
(43) Pub. Date: Jul. 17, 2008

(54) LOW POWER METHODS FOR PRESSURE WAVEFORM SIGNAL SAMPLING USING IMPLANTABLE MEDICAL DEVICES

(76) Inventors: Paul J. Huelskamp, St. Paul, MN (US); Wangcai Liao, Cary, NC (US); Keith R. Maile, New Brighton, MN (US); Binh C. Tran, Minneapolis, MN (US)

Correspondence Address: FAEGRE & BENSON, LLP;32469, 2200 WELLS FARGO CENTER, 90 SOUTH SEVENTH STREET, MINNEAPOLIS, MN, 55402-3901, US

(21) Appl. No.: 12/013,325

(22) Filed: Jan. 11, 2008

Related U.S. Application Data
(60) Provisional application No. 60/884,855, filed on Jan. 12, 2007.

(51) Int. Cl. A61B 5/0205

(52) U.S. Cl. 600/484

(57) ABSTRACT

Systems and methods for reducing power consumption in implantable medical devices (IMDs) in which an IMD implantable in an artery monitors blood pressure. A master device monitors a physiological signal, such as respiratory cycle, and instructs the IMD to take blood pressure measurements over a sampling interval, the duration of which is determined by the master device based on the monitored physiological signal. The master device may determine an end-expiration point of the respiratory cycle and send synchronization information to the IMD to further shorten the sampling interval by coinciding the sampling interval with the end expiration point of the respiratory cycle. The IMD may further conserve power by including processing abilities to collect and/or transmit only a subset of data representing the blood pressure signal, for example, systolic, diastolic, and/or mean blood pressure signal values. The blood pressure readings, once taken, may be transferred to the master device.

### CROSS-REFERENCE TO RELATED APPLICATION

This application claims the benefit of U.S. Provisional Patent Application Ser. No. 60/884,855, filed on Jan. 12, 2007, and entitled, “Low Power Methods for Pressure Waveform Signal Sampling Using Implantable Medical Devices,” which is incorporated herein by reference in its entirety.

### TECHNICAL FIELD

Various embodiments of the present invention generally relate to implantable medical devices. More specifically, embodiments of the present invention relate to low power methods for pressure waveform signal sampling using implantable medical devices.

### BACKGROUND

Medical devices can be implanted in the bodies of patients for various purposes including therapy delivery and monitoring of one or more internal states of the patient. Examples of internal patient states include blood pressure, temperature, and the like. In many cases, implantable medical devices (IMDs) are intended to remain indefinitely within the patient. Because the IMDs have a limited supply of power, battery conservation is often desirable to extend use.

Some current methods for monitoring states within a patient are performed based on a fixed period of sampling to ensure that the desired measurements are appropriately recorded. However, if frequent measurements are needed, the power supply within the IMD may be quickly drained necessitating device replacement or recharging. Replacing or recharging IMDs within the patient's body can be time consuming, inconvenient, and may reduce the quality of the patient's life. As such, the limited power resources in an IMD should be used as efficiently as possible to reduce interventions needed to keep the IMD functioning.

### SUMMARY

Systems and methods are described for low power pressure waveform signal sampling using implantable medical devices. According to various embodiments, an implantable medical device (IMD) configured for implantation in a pulmonary artery of a patient to monitor blood pressure is disclosed. The IMD, according to at least one embodiment, includes a battery, a memory, a processor, a sampling mode module, a synchronization module, a sensor module, and a communications module.

According to various embodiments, the synchronization module determines a timing signal that specifies a sampling interval based on a received physiological signal. The received physiological signal may be a heart rhythm, respiratory rhythm, minute ventilation, breath rate, posture, or the like. The pressure sensor module, according to various embodiments, receives the timing signal from the synchronization module and makes blood pressure readings according to the timing signal.

In one or more embodiments, the timing signal specifies the length of the sampling interval based on the received physiological signal. The timing signal may also specify the start of the sampling interval based on the received physiological signal in various embodiments. For example, in some embodiments, the timing signal indicates a start time and length of a sampling interval to allow blood pressure readings or measurements to occur during end-expiration of a ventilation cycle of the patient.

In some embodiments, the system is capable of generating a battery status signal indicating a current level of charge remaining. The sampling mode module may use the status signal indicating the current level of charge of the battery to determine an appropriate sampling mode. In other embodiments, the sampling mode module may use a signal from a master device to determine the sampling mode. Examples of information included in the sampling mode of the pressure sensor module includes, but is not limited to, sample rate, data compression rate, and measurement frequency.

The memory within some embodiments may be used to store blood pressure readings taken by the pressure sensor which may be communicated via communications module. In some embodiments, the processor may be used to generate statistics about the blood pressure readings and cause the statistics to be communicated to a requesting or commanding device via a communications module. In other embodiments, the communications module may select the blood pressure measurement closest to the end-expiration of a ventilation cycle of the patient and then transmit the selected blood pressure measurement to a second IMD.

According to some embodiments of the present invention, an implantable medical device system with reduced power consumption includes a master device and a slave implantable medical device. The master device may be configured to receive a physiological signal (e.g. a respiratory cycle signal) of a patient, to determine a sampling interval duration based on the physiological signal, and to send an instruction containing the sampling interval duration. The slave implantable medical device may be configured to receive the instruction containing the sampling interval duration, to sense another physiological signal (e.g. a blood pressure signal), and to record one or more data points from the other physiological signal over the sampling interval duration, according to embodiments of the present invention.

While multiple embodiments are disclosed, still other embodiments of the present invention will become apparent to those skilled in the art from the following detailed description, which shows and describes illustrative embodiments of the invention. As will be realized, the invention is capable of modifications in various aspects, all without departing from the scope of the present invention. Accordingly, the drawings and detailed description are to be regarded as illustrative in nature and not restrictive.

### BRIEF DESCRIPTION OF THE DRAWINGS

In the Figures, similar components and/or features may have the same reference label. Further, various components of the same type may be distinguished by following the reference label with a second label that distinguishes among the similar components. If only the first reference label is used in the specification, the description is applicable to any one of the similar components having the same first reference label irrespective of the second reference label.

FIG. 1 illustrates an exemplary environment with which embodiments of the present invention may be utilized;

FIG. 2 illustrates a block diagram of components of an implantable medical device which may be used in accordance with one or more embodiments of the present invention;

FIG. 3 illustrates an exemplary system diagram in accordance with various embodiments of the present invention;

FIG. 4 illustrates a flowchart containing exemplary operations which may occur in accordance with some embodiments of the present invention;

FIG. 5 illustrates an exemplary sampling interval in accordance with one or more embodiments of the present invention;

FIG. 6 illustrates a flowchart containing exemplary operations which may be performed in accordance with various embodiments of the present invention;

FIG. 7 illustrates an exemplary sampling interval in accordance with some embodiments of the present invention;

FIG. 8 illustrates an exemplary sampling interval in accordance with various embodiments of the present invention;

FIGS. 9A-9B illustrate a modified command protocol which may be used in accordance with some embodiments of the present invention;

FIG. 10 illustrates a discriminating operation of one or more embodiments of the present invention;

FIG. 11 is a flowchart containing exemplary operations in accordance with one or more embodiments of the present invention; and

FIG. 12 illustrates an exemplary computer system which may be used in conjunction with one or more embodiments of the present invention.

While the invention is amenable to various modifications and alternative forms, specific embodiments have been shown by way of example in the drawings and are described in detail below. The intention, however, is not to limit the invention to the particular embodiments described. On the contrary, the invention is intended to cover all modifications, equivalents, and alternatives falling within the scope of the invention as defined by the appended claims.

### DETAILED DESCRIPTION

Various embodiments of the present invention generally relate to implantable medical devices. More specifically, various embodiments of the present invention relate to low power methods for pressure waveform signal sampling using implantable medical devices.

Embodiments of the present invention may be used to detect and monitor physiological signals within a patient though the use of one or more implantable medical devices (IMD). Many IMDs are intended for permanent implantation within a patient. Due to their permanent nature, the battery powering the IMD is preferably appropriately sized to ensure significant product life with minimal intervention. However, as devices have become smaller, battery sizes have also decreased, making capacity, efficiency, recharge time, and the time interval between recharges important factors in device management.

According to one embodiment, a method of operation of an IMD implanted within the pulmonary artery of a patient uses a physiological signal, such as minute ventilation or heart rate, as an indication of how long the sample interval should be to appropriately sample a blood pressure measurement in the ventilation cycle. For example, a typical full period for a breath in a respiratory cycle may range from three to seven seconds. The minute ventilation may be used to determine the current length of the average breath of the patient. According to one embodiment, a blood pressure measurement occurs during end-expiration of a ventilation cycle of the patient. Because the average length of the breath is known, an appropriately sized sample interval may be used which ensures that blood pressure samples will overlap the end-expiration of a ventilation cycle.

According to another embodiment of the present invention, once the respiration rate is known, the blood pressure measurement cycle may be used to synchronize the measurement cycle with the end-expiration of the ventilation cycle. In one embodiment, the crossover point in the minute ventilation signal is used to determine when to take the blood pressure measurements. The measurements may then be transmitted to a second device for additional processing.

In some embodiments of the present invention, less than all of the measurements are transmitted to the second device. In one embodiment, the IMD may pick off and transmit only a subset of the data points and/or processed data points from the measurement cycle. This reduces the power consumption required to transmit the measurement data.

In the following description, for the purposes of explanation, numerous specific details are set forth in order to provide a thorough understanding of embodiments of the present invention. It will be apparent, however, to one skilled in the art that embodiments of the present invention may be practiced without some of these specific details.

Embodiments of the present invention may be provided as a computer program product which may include a machine-readable medium having stored thereon instructions which may be used to program a computer (or other electronic device) to perform a process. The machine-readable medium may include, but is not limited to, floppy diskettes, optical disks, compact disc read-only memories (CD-ROMs), and magneto-optical disks, ROMs, random access memories (RAMs), erasable programmable read-only memories (EPROMs), electrically erasable programmable read-only memories (EEPROMs), magnetic or optical cards, flash memory, or other type of media/machine-readable medium suitable for storing electronic instructions. Moreover, embodiments of the present invention may also be downloaded as a computer program product, wherein the program may be transferred from a remote computer to a requesting computer by way of data signals embodied in a carrier wave or other propagation medium via a communication link (e.g., a modem or network connection).

While, for convenience, some embodiments of the present invention are described with reference to blood pressure measurements from an IMD implanted within the pulmonary artery, embodiments of the present invention are equally applicable to various other physiological measurements and IMD devices.

For the sake of illustration, various embodiments of the present invention have herein been described in the context of computer programs, physical components, and logical interactions within electronic and software components of IMDs and modern networks. Importantly, while these embodiments describe various aspects of the invention in relation to IMD electronics, software, and programs, embodiments of the method and apparatus described herein are equally applicable to other systems, devices, and networks as one skilled in the art will appreciate. As such, the illustrated applications of the embodiments of the present invention are not intended to be limiting, but instead exemplary. Other systems, devices, and networks to which embodiments of the present invention are applicable include, but are not limited to, other types of sensory systems and networks and computer devices and systems. In addition, embodiments are applicable to all levels of sensory devices from a single IMD with a sensor to large networks of sensory devices and computers.

Terminology

Brief definitions of terms, abbreviations, and phrases used throughout this application are given below.

The terms “connected” or “coupled” and related terms are used in an operational sense and are not necessarily limited to a direct physical connection or coupling. Thus, for example, two devices may be coupled directly, or via one or more intermediary media or devices. As another example, devices may be coupled in such a way that information can be passed therebetween, while not sharing any physical connection with one another. Based on the disclosure provided herein, one of ordinary skill in the art will appreciate a variety of ways in which connection or coupling exists in accordance with the aforementioned definition.

The phrase “implantable medical device” generally refers to any device which may be implanted within a living being. Accordingly, an implantable medical device may be passive and only monitor events or an implantable medical device may have a therapeutic function such as electrical stimulation or drug delivery, for example.

The phrases “in one embodiment,” “according to one embodiment,” and the like generally mean the particular feature, structure, or characteristic following the phrase is included in at least one embodiment of the present invention, and may be included in more than one embodiment of the present invention. Importantly, such phases do not necessarily refer to the same embodiment.

If the specification states a component or feature “may”, “can”, “could”, or “might” be included or have a characteristic, that particular component or feature is not required to be included or have the characteristic.

The term “responsive” includes completely and partially responsive.

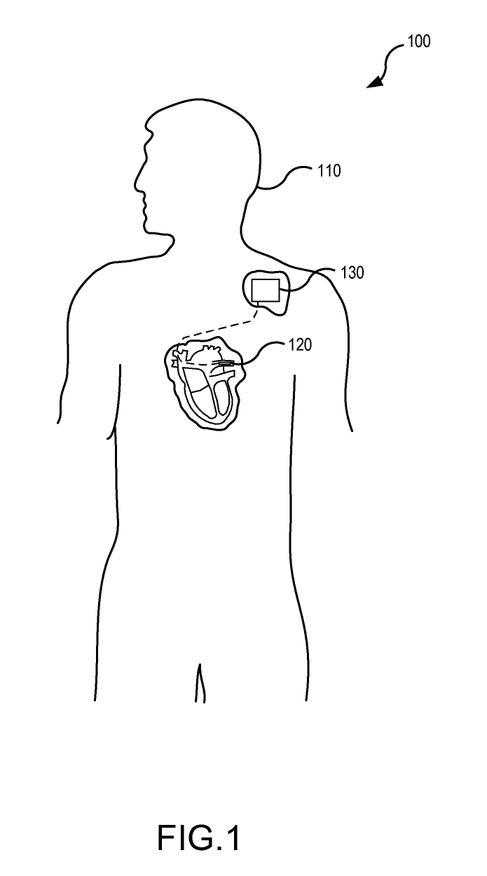

FIG. 1 illustrates an exemplary environment 100 with which embodiments of the present invention may be utilized. According to various embodiments of the present invention, an implantable medical device (IMD) 120 may be implanted within a patient 110. In some cases, patient 110 may be a human. In other cases, patient 110 may be a pet such as a dog, cat or other animal, for example.

In accordance with various embodiments, an IMD 120 may be implanted within the pulmonary artery, other vessels, or within the heart of the patient 110. According to embodiments of the present invention, IMD 120 may be used to monitor one or more physiological signals and/or perform a therapeutic function. In some instances, IMD 120 may be difficult, if not impossible, to remove once it is implanted within the patient 110. In some embodiments, IMD 120 may be implanted with an understanding that the IMD will never be removed from the patient 110. According to one embodiment, IMD 120 is capable of measuring ambulatory blood pressure of patient 110 and may be required to make multiple measurements throughout the day. For example, in one embodiment, IMD 120 may be configured to record sixty-four measurements per day. Other embodiments allow for IMD 120 to record more or less than sixty-four measurements per day.

Once IMD 120 makes a measurement, some or all of the measurement data are then transmitted to a second device 130. The transmission of the data may occur as the measurements are taken or the measurements may be stored and transmitted at a later time. According to various embodiments, second device 130 may be another IMD or an external device which has a larger power source (e.g. greater battery power) and/or more computational power than IMD 120. According to some embodiments, second device 130 is a pulse generator unit. According to some embodiments of the present invention, IMD 120 is communicably coupled with second device 130 such as, for example, by a wire connection and/or a wireless connection.

By customizing the way in which IMD 120 collects measurements and/or the way in which IMD 120 communicates with second device 130 to exchange measurements and/or instructions, the power consumption of IMD 120 may be reduced, permitting an increased battery life for IMD 120. For example, rather than recording blood pressure measurements over a large sample interval intended to cover the longest expected respiration cycle or the longest statistical respiration cycle, IMD 120 may be customized to shorten the sample interval based on an actual observation of the particular respiration cycle in the patient 110 in which IMD 120 is implanted.

Because a blood pressure measurement taken at the end expiration point of a respiration cycle may be particularly medically useful, further power savings for IMD 120 may be achieved by customizing IMD 120 to record and/or transmit to second device 130 only blood pressure measurements taken at or near the end expiration of the respiratory cycle of patient 110, according to embodiments of the present invention. And because a systolic and/or peak blood pressure measurement may be particularly medically useful, further power savings for IMD 120 may be achieved by customizing IMD 120 to record and/or transmit to second device 130 only systolic and/or peak blood pressure measurements from the blood pressure signal, according to embodiments of the present invention.

According to some embodiments, second device 130 is the master device and IMD 120 is the slave device, which permits minimization of the computational power, memory capacity, and periphery sensing capability of IMD 120 by permitting IMD 120 to receive and execute measurement, measurement mode, and/or other parameter-related instructions from second device 130. According to other embodiments, some basic computational features may be performed by IMD 120, while other calculations and/or instructions may be processed and sent by second device 130.

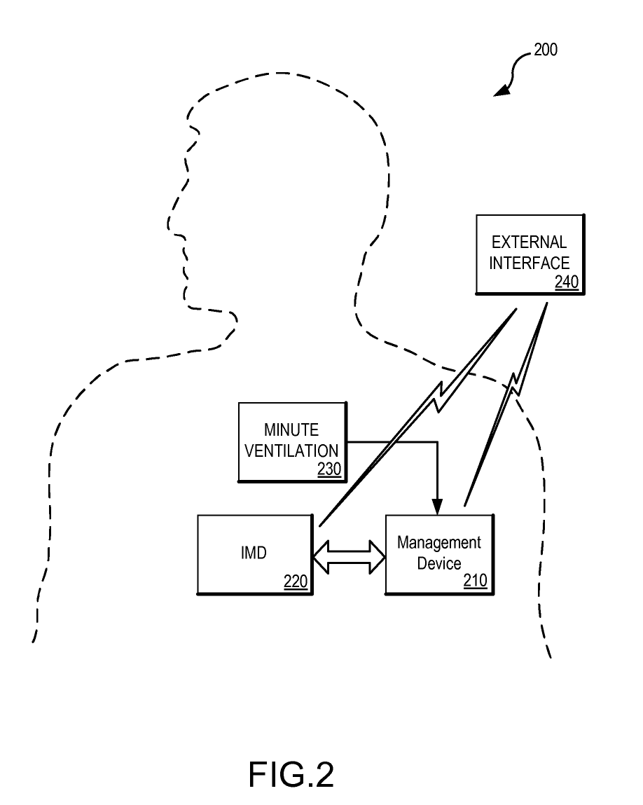

FIG. 2 illustrates an exemplary system diagram 200 in accordance with various embodiments of the present invention. According to various embodiments of the present invention, IMD 220 may be part of a monitoring system. As illustrated in FIG. 2, an internal management device 210 may govern the operation of IMD 220. In some embodiments, internal management device 210 may be a pacemaker, defibrillator, pulse generator, another sensor, or the like. Internal management device 210 may communicate a desired sampling mode to the IMD 220. In some embodiments, IMD 220 communicates measurements to internal management device 210 and/or external interface 240.

Embodiments of the present invention also allow for management device 210 to receive one or more additional physiological signals to determine the desired sampling mode or sampling parameters for the primary signal to be measured by IMD 220. In the embodiment depicted in FIG. 2, a minute ventilation module 230 senses the respiratory signal and transmits a minute ventilation signal to management device 210. According to other embodiments, minute ventilation module 230 may be located within management device 210 and sense the respiratory signal from within management device 210. This signal may be used, in accordance with various embodiments, to determine sample frequency and/or interval duration based on the respiration rate, and may be used as a trigger for indicating when the sample interval may begin on IMD 220.

In one embodiment, external interface 240 may communicate directly with internal management device 210 to extract data and provide management instructions such as measurement schedule and the like. External interface 240, according to some embodiments, may include a graphical user interface which allows a doctor or patient to retrieve data, request measurements, or set modes of IMDs within the patient so that the batteries may be recharged. In other embodiments, external interface 240 may be a computer which can be used for processing data, generating reports, setting IMD modes, and other functions.

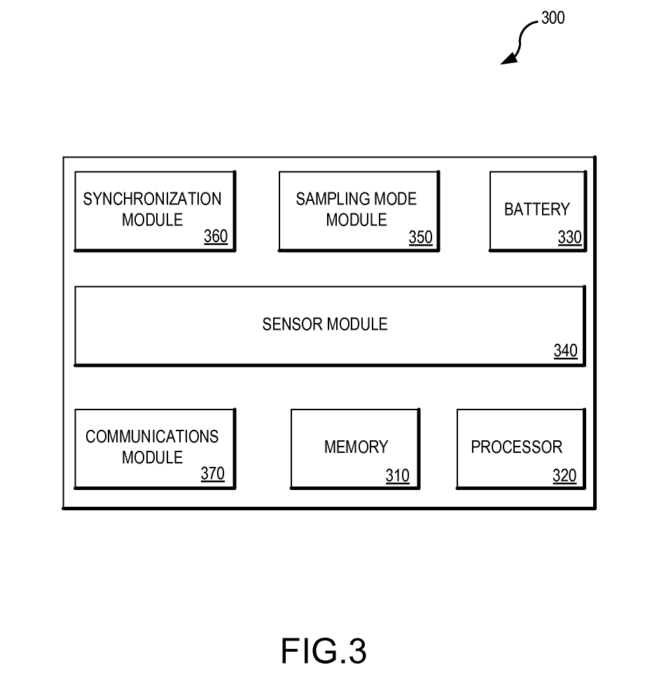

FIG. 3 illustrates a block diagram of components of an implantable medical device 300 which may be used in accordance with one or more embodiments of the present invention. According to various embodiments, IMD 300 may include a memory 310, a processor or controller 320, a power source 330, a sensor module 340, a sampling module 350, a synchronization module 360, and a communications module 370. According to some embodiments, sensor module 340, sampling module 350, synchronization module 360, and communications module 370 may be implemented in hardware, software or a combination thereof. Moreover, while various components have been separated in FIG. 3 for discussion purposes, in one or more embodiments of the present invention some of these elements may be combined, absent, or duplicated. Furthermore, some or all of these elements may be distributed between an IMD 300 and a second device 130; for example, elements which require a higher power consumption (e.g. more computational power, memory storage, and communications capacity) may be located in second device 130 in order to minimize power consumption in IMD 300, according to some embodiments of the present invention.

In various embodiments, sensor module 340 within IMD 300 may be used to measure blood pressure signals. Measurements of the signal may be stored in memory 310 and then transmitted to a secondary device through communications module 370. According to some embodiments, a subset of the data taken by sensor module 340 or values computed from the data (e.g. minimum, maximum, mean) may be transmitted. According to other embodiments, all of the data taken by sensor module 340 may be transmitted. In one embodiment, sensor module 340 and sampling mode module 350 operate according to instructions received by processor 320 and/or synchronization module 360 from second device 130.

For example, in one embodiment, processor 320 may be running instructions received from a master device 130 which indicate the time of day when sensor module 340 should perform measurements and which indicate a sampling rate. In other embodiments, instructions on processor 320 may be monitoring for an event or trigger to indicate when the sensor module 340 should be activated to receive measurements; for example, such an event or trigger may be one or more instructions received from a master device 130. In addition, in some embodiments, the instructions running on processor 320 may be used to determine the appropriate length of the sampling interval used by sensor module 340; such instructions may be based on instructions received from a master device 130. In one embodiment, an indication of a sampling mode may be commanded for and/or received by sampling mode module 350. One example of a sampling mode is a fixed sampling interval. Another example is a dynamic sampling interval which may be determined in part by one or more physiological signals such as heart beat, posture, and/or minute ventilation, for example.

In some embodiments, a sampling mode may include a synchronization mode which aligns the sampling interval so that the desired point where a measurement should be taken is likely to fall within the sampling interval. According to various embodiments, when a synchronization mode is requested or commanded, synchronization module 360 may be used to obtain the triggering event. For example, synchronization module 360 synchronizes the sensor module 320 measurement cycle with the respiratory cycle of the patient, according to some embodiments. Such synchronization may, for example, involve overlapping the sample interval with the end-expiration for either a spontaneous ventilation or for a mechanical ventilation cycle. In some embodiments, synchronization module may perform some processing or filtering on the raw triggering data to prevent false triggers from occurring.

Another example of a sampling mode is a minimal transfer mode. When the minimal transfer mode is requested or commanded, only a subset of the data collected by sensor module 340 will be transmitted to a second device using communications module 370. According to one embodiment, the data may be processed, filtered, and/or compressed before sending. In another embodiment, only selected points may be sent through communications module 370. For example, according to one embodiment, only diastolic and systolic values sampled during a measurement cycle may be sent to a second device. According to another embodiment, approximate diastolic and systolic values sampled during a measurement cycle may be sent to a secondary device via the communications module 370. In other cases, one or more statistics about the diastolic or systolic values during the measurement cycle may be sent to the second device. The statistics transmitted to the second device may include mean value, average value, standard deviation, range, maximum value, minimum value, and the like.

According to one or more embodiments, the sampling mode may change over time, including from one measurement interval to the next. In some embodiments, the sampling mode may be determined by factors such as remaining power in battery 330, the desired task, requested accuracy, availability of a synchronization signal, as well as other factors.

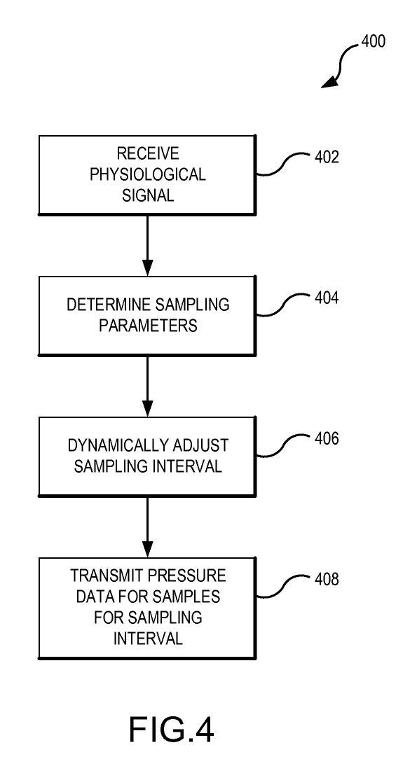

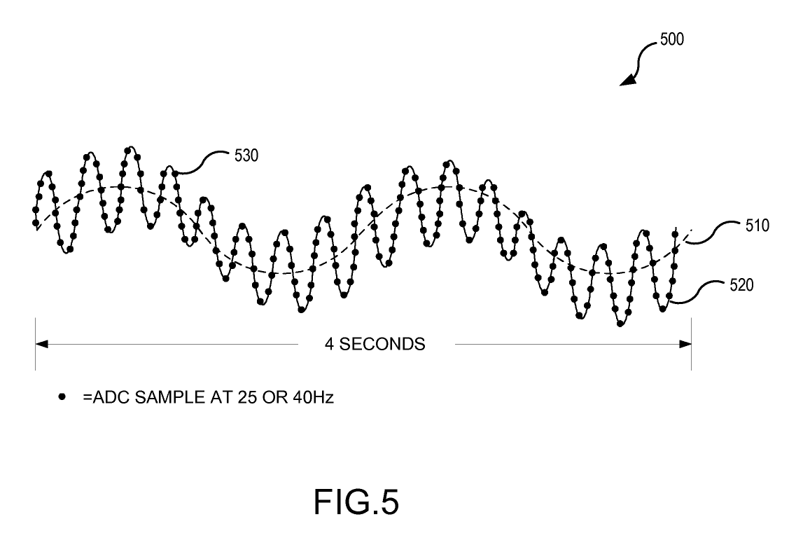

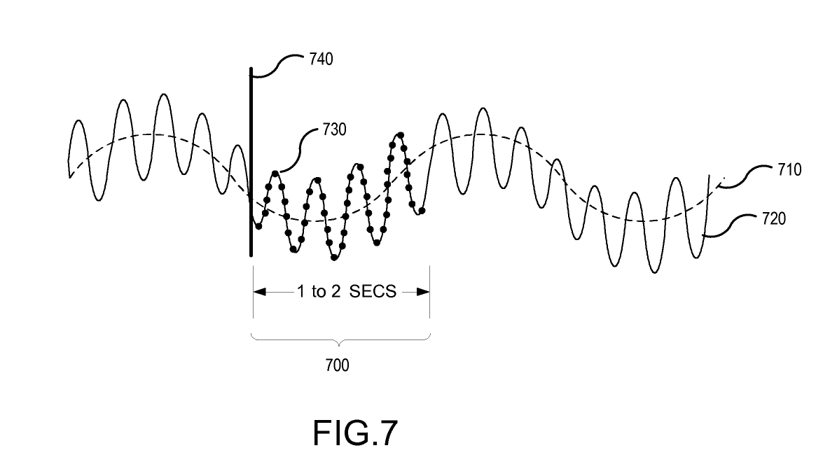

FIG. 4 illustrates a flowchart 400 containing exemplary operations which may occur in accordance with some embodiments of the present invention. According to one embodiment of the present invention, such exemplary operations occur within management device 130. At receiving operation 402, a physiological signal is received. The physiological signal may be, for example, a respiratory or cardiac rhythm, their subsets including a minute ventilation signal or a heart rate signal or the like. According to various embodiments, determination operation 404 determines the sampling parameters based, at least in part, on the received physiological signal. FIGS. 5 and 7 illustrate examples of such determinations according to embodiments of the present invention. Alternatively the signal may be a non-physiological signal such as, for example, time of day.

In one embodiment, adjustment operation 406 dynamically adjusts the sampling interval used by the IMD based on the determined sampling interval from determination operation 404. Once the blood pressure data is gathered, processed, and/or filtered, a transmission operation 408 transmits the blood pressure data to a second device.

Without monitoring a physiological signal, such as minute ventilation, heart rate, or others, and determining the breathing rate (e.g. minimum, maximum, mean), an unnecessarily long measurement interval may often be used to ensure that the measurement includes the desired points. For example, the average breath in a typical respiratory cycle is three to seven seconds. According to some embodiments of the present invention, the sample length is approximately twice the length of the longest possible breath in order to ensure that a desired blood pressure data point, such as, for example, the systolic pressure at end respiration, is captured. In one embodiment, the sample length is set to approximately fifteen seconds, and is possibly greater than twice the duration required to capture such a data point.

FIG. 5 illustrates an exemplary sampling interval 500 in accordance with one or more embodiments of the present invention. For example, if the average breath length is about two seconds, then as depicted in FIG. 5, the sample interval 500 may be reduced to four seconds. Consequently, this reduces the number of samples needed to obtain data points of interest which, according to some embodiments, reduces operating power of IMD 120 by approximately 60% from a baseline sample duration of fifteen seconds.

Sample interval 500 depicts an exemplary blood pressure waveform 520 superimposed upon an exemplary respiratory signal 510 of a patient. An IMD in accordance with one embodiment of the present invention is able to take measurements 530 at a requested or determined sampling rate over the determined sampling interval duration. According to various embodiments, the sampling rate may be varied depending on the frequency of the cardiac cycle. In some embodiments, the sampling rate ranges from twenty-five to forty hertz (Hz). In other embodiments, the sampling rate may be higher than forty hertz or lower than twenty-five hertz.

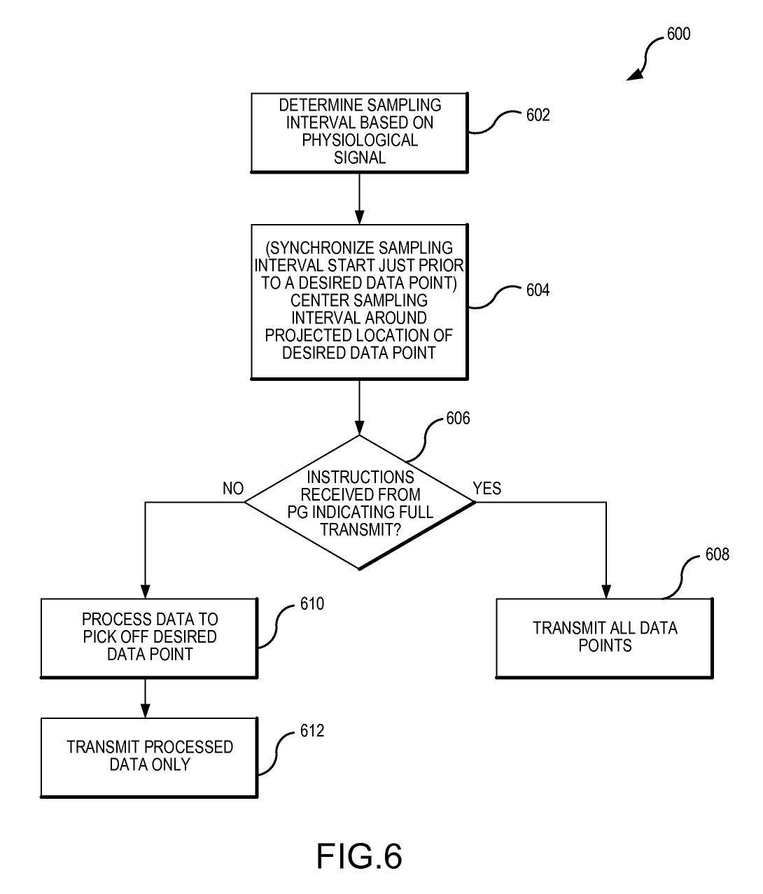

FIG. 6 illustrates flowchart containing exemplary operations 600 which may be performed in accordance with various embodiments of the present invention. According to various embodiments, the physiological signal may be used predict the location of a desired data point. For example, in one embodiment, the desired data point is the blood pressure measurement occurring at end-expiration of a ventilation cycle. The sample time interval may be accordingly reduced based on this prediction.

In accordance with one or more embodiments, a determination operation 602 determines the sampling interval based on a received physiological signal. This signal may be filtered, processed, or used in conjunction with other signals to predict where a sample point of interest is likely to occur. The reduced sample interval may then be centered about the projected location of the desired data point of interest by centering operation 604. Multiple blood pressure samples may be taken during this interval in accordance with one embodiment.

If the IMD has received one or more instructions from a master device commanding a full transmission of the data at decision operation 606, then all of the data points may be transmitted to a second device by operation 608. According to various embodiments, this transmission may occur simultaneously with data recordation, at the end of the sample interval, as the memory usage passes a certain threshold, after some filtering occurs, after an event occurs (such as a request from a device), and/or after a fixed time delay, for example.

If the IMD has not received instructions from a master device commanding a full transmission of the data at decision operation 606, then process operation 610 may occur. Process operation 610, according to one or more embodiments, processes the data and selects or “picks off” desired data points. For example, process operation 610 may select only the systolic and/or diastolic values, or approximations thereof, from the pressure measurements. Once the data has been processed it may then be transmitted to a second device via transmit operation 612. Those of ordinary skill in the art, based on the disclosure provided herein, will appreciate that embodiments of the present invention may compress, encrypt, or prepare the data for transfer by error correcting bits prior to transferring the data.

FIG. 7 illustrates an exemplary sampling interval 700 in accordance with some embodiments of the present invention. An exemplary blood pressure waveform 720 is shown superimposed upon an exemplary respiratory signal 710 of a patient. According to various embodiments of the present invention, it may be desirable to take the blood pressure measurement at an end-expiration point of the respiratory cycle. Using exemplary operations as described with respect to FIG. 6, the sampling interval may be centered around the end-expiration point of the ventilation cycle. This may be done, according to some embodiments, by synchronizing the pressure measurements 730 with a triggering event 740 such as the minute ventilation cross-over point or other corresponding signal received from a master device. According to some embodiments of the present invention, the minute ventilation cross-over point is an interrupt received by the master device 130, such as a pulse generator. A measurement by IMD 120 may thus be timed based on the interrupt; according to some embodiments of the present invention, the precision of IMD 120 in taking measurements based on the interrupt is approximately one hundred milliseconds.

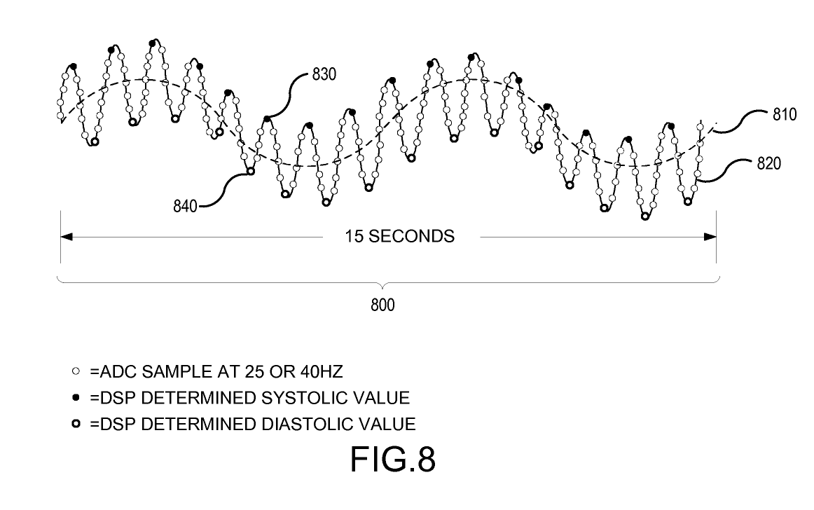

FIG. 8 illustrates an exemplary sampling interval 800 in accordance with various embodiments of the present invention. An exemplary respiratory signal 810 is depicted. Superimposed upon the respiratory signal 810 is an exemplary blood pressure waveform 820 of a patient. A limited sampling solution in one or more embodiments moves the diastolic and/or systolic value detection algorithm normally residing on a secondary device 130, 210, such as a pulse generator (PG), to an IMD 120, 220. As a result, the IMD 120, 220 may return only the diastolic 840 and/or systolic 830 values of the blood pressure cycle 820 according to embodiments of the present invention. Consequently, the IMD 120, 220 takes measurement data at a significantly lower frequency, transmits significantly less data to the secondary device 130, 210, and turns off power consuming components. Examples of power consuming components which may be turned off include, but are not limited to, acoustic communication modules, fast clocks and the like.

According to various embodiments, the measurements taken by the sensor module may be queued up for transfer to a second device and/or statistically processed and the statistics transferred. In some embodiments, the information may be encoded with error correcting bits before transmission. According to various other embodiments, the measurements may be transferred as acquired. One advantage to queuing up the data points and then transferring them to the second device is that the sampled values can be re-transmitted if the received signal has errors.

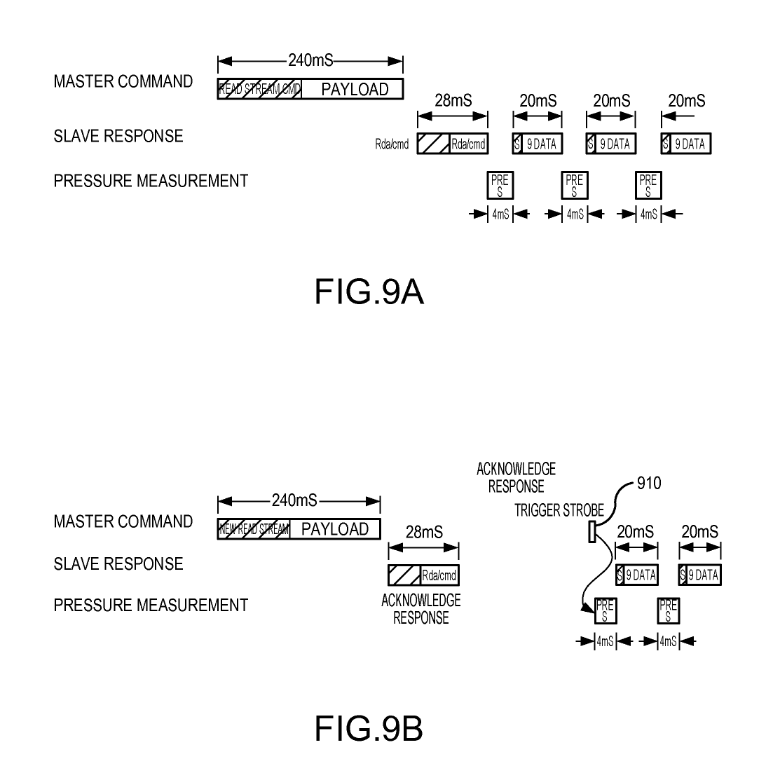

FIGS. 9A-9B illustrate a modified command protocol which may be used in accordance with some embodiments of the present invention. As previously described, embodiments of the present invention may be used to reduce the amount of power consumed in IMD 300. Variations of the embodiments described may be used separately or jointly and to varying degrees to enable a variety of measurement and power options.

A dynamic sampling interval method limits the time associated with the actual measurement of the physiological signal. According to various embodiments, memory store 310 may be a simple programmable register and can be used to store the measurements. Five bits at 1-second resolution will allow measurements between one and thirty-two seconds to be stored. The programmable register may be programmed via the pressure measurement command and/or a programmable register in the memory map that can be retained from session to session (e.g. stored in EEprom).

As illustrated in FIG. 9A, when synchronizing the beginning of the stream measurement, one approach is to utilize an existing “Read Stream” command, which takes approximately two-hundred forty milliseconds to send and approximately another thirty milliseconds before the first measurement is taken, according to embodiments of the present invention.

Alternatively, according to various embodiments, a new command accepting a “trigger” 910 to start a measurement may be used as shown in FIG. 9B. The two-hundred forty milliseconds of the command being sent and acknowledgment are removed from the trigger timing giving one advantage over the existing “Read Stream” command in FIG. 9A. For example, when a physiological signal is received by master device 130 indicating that the end expiration point of a respiration cycle has occurred or is about to occur, master device 130 may then command IMD 120, 220 to take one or more pressure measurements. The command structure depicted in FIG. 9B may, according to some embodiments of the present invention, eliminate the two hundred forty millisecond delay associated with the command structure depicted in FIG. 9A, permitting the pressure measurements to be taken more immediately after the time IMD 120 is instructed to do so. One disadvantage to such an approach is that such a new command introduces another command/response structure to the IAC protocol.

In accordance with various embodiments of the present invention, one or more data compression, filtering, and/or down sampling schemes may be used. According to one embodiment, the streaming blood pressure data may be filtered or compressed into specific data points that can be read out individually after the measurement period has expired. An exemplary design is described, but based on the disclosure provided herein, those of ordinary skill in the art will appreciate possible alternatives and the multiple variables that modify the operation of the logic.

In one embodiment, the primary purpose of the sensor module 340 within IMD 300 is to accurately provide a measurement of the blood pressure within the artery in which the IMD is placed. Because the IMD may be implanted via a catheter, minimizing the size of the sensor module may be desirable. In addition, because the IMD cannot typically be explanted, it may be desirable to maximize the longevity of any IMD. Data compression algorithms may be used in accordance with various embodiments of the present invention. The use of one or more data compression algorithms to accurately provide a measurement using as little power as possible in the smallest possible area can be useful in reducing power consumption. Embodiments of the present invention balance increasing accuracy with the size or area of the IMD 220, 300.

For example, one or more of the following design constraints may be used in the design of components of IMD 220: 1) a maximum of one hundred fifty beats per minute may be assumed, and the sampling interval determined based on the respiration period; 2) only the peak and valley within a single heart cycle will be stored, accumulated, and/or counted (i.e., only the diastolic and systolic values and not mid points will be recorded); and 3) the design of the IMD 300 will be 5,000 gates or less.

In accordance with one embodiment of the present invention, a component of the data compression algorithm may “pick off” the peak (maximum) and valley (minimum) data points within a single heart cycle. In some embodiments, a simple “greater than” or “less than” comparator may be used. This simple comparator would work effectively if the signal had no notches, or false peaks or valleys, within a waveform. However, a physiological signal is very likely to have multiple turns within a heart cycle and a certain amount of noise in the physiological signal.

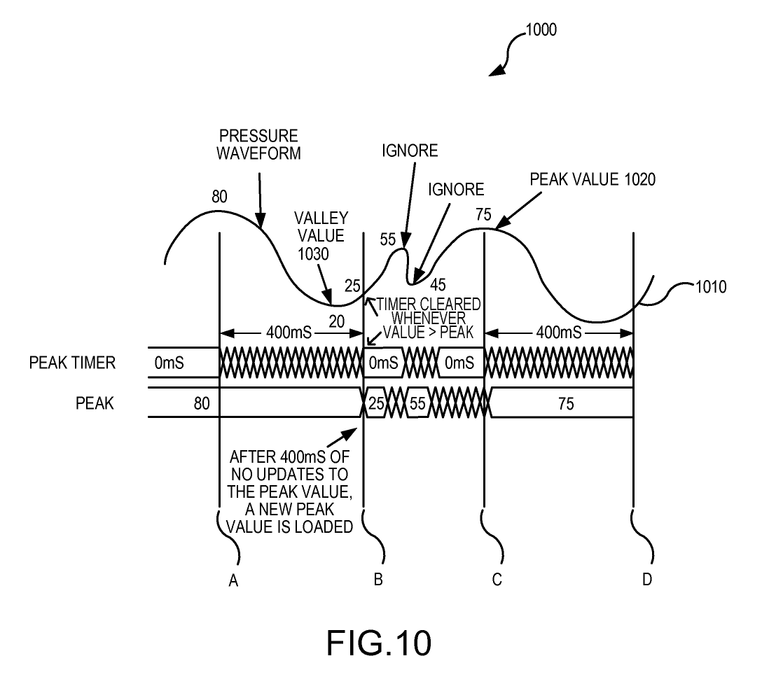

FIG. 10 illustrates a discriminating operation 1000 of one or more embodiments of the present invention. According to some embodiments, the logic for performing such a discriminating operation 1000 may reside on IMD 120, 220, 300; although the performance of such logic may consume additional power from the battery of the IMD, doing so may use less power than that required to transmit a larger data set to a master device. In accordance with one embodiment, a time based method to determine the peak/valley value of a signal 1010 may be used. In one embodiment, a minimum time interval may be used to track a peak 1020 or valley 1030. For example, with a maximum heart rate of one-hundred fifty beats per minute (bpm), a four-hundred millisecond sampling interval may be used. In an embodiment, a peak 1020 (or valley 1030) would be monitored. Every time the peak was updated (current value>peak value), a four-hundred milliseconds timer would be cleared. A new peak interval would start when the four-hundred milliseconds timer expires.

For example, in FIG. 10, a potential peak is detected at a value of eighty at point A. A temporary value is set to the potential peak value of eighty and a four-hundred millisecond timer begins. Another potential peak value is not found during this four-hundred millisecond period as illustrated between points A and B. According to one embodiment, when the timer expires at point B a new peak value may be loaded into the temporary value. In one embodiment, the value loaded at the end of the expiration of the timer is the value of the physiological signal at that time (or the next sample time). As illustrated in FIG. 10, this value is twenty-five.

The values of the signal are monitored and when a new potential peak is determined (i.e., current value>peak value), the temporary value is replaced. In FIG. 10, a value of fifty-five occurs between points B and C, the temporary value is set to fifty-five, and a four-hundred millisecond timer begins. If, however, as illustrated in FIG. 10 at point C another potential peak is detected before the expiration of the four-hundred millisecond timer, the timer is reset and the temporary value is replaced with the new potential peak value 1020 of seventy-five. Consequently, the potential peak at fifty-five is determined to be a false systolic value and is not recorded. Between points C and D on FIG. 10, no additional potential peaks are detected before the expiration of the timer.

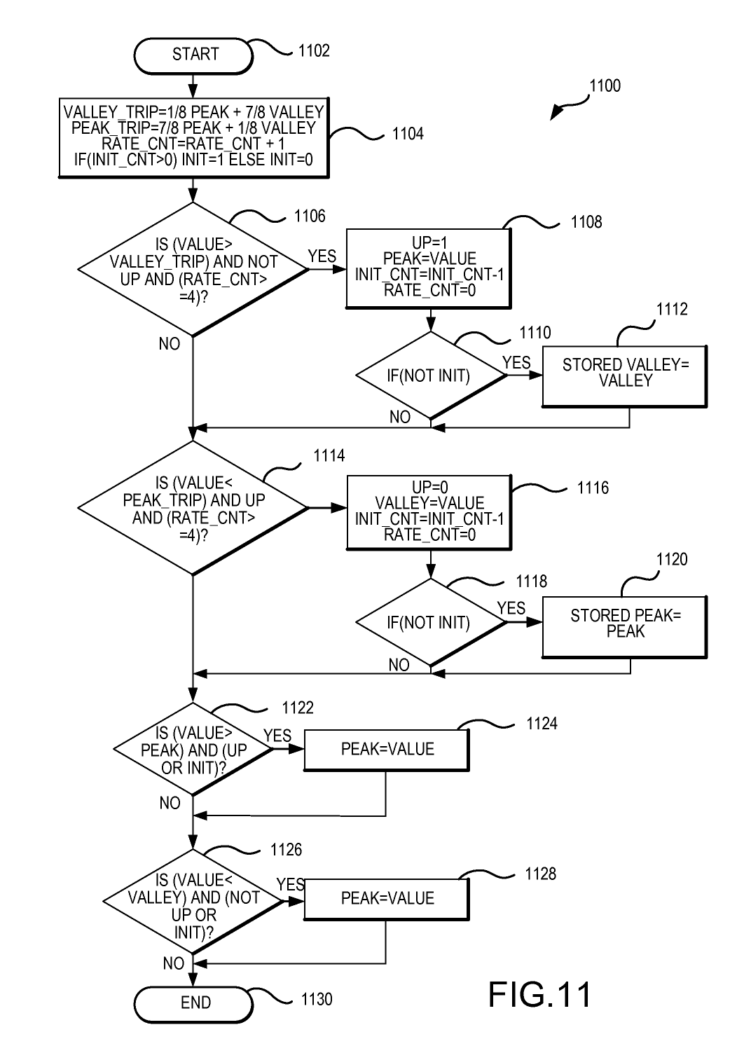

According to one embodiment, a threshold between the current peak and valley is used to determine when a peak or valley value should be tracked. FIG. 11 shows an exemplary flow chart 1100 illustrating one such method in accordance with embodiments of the present invention.

The algorithm starts at starting block 1102. From here, computation operation 1104 computes a valley trip value, a peak trip value and a rate count. According to various embodiments, the valley trip value and the peak trip values are weighted averages of the peak and valley values which have been determined. In one embodiment, the following weighted averages shown in Eq. 1 and Eq. 2 may be used.

VALLEY_TRIP=⅛ PEAK+⅞ VALLEY (Eq. 1)

PEAK_TRIP=⅞ PEAK+⅛ VALLEY (Eq. 2)

Also, according to various embodiments, an initialization run of the algorithm may be performed. If the current run is an initialization run, then a variable INIT is set to 1, else INIT is set to 0.

At decision operation 1106, a determination is made whether the VALUE is greater than the valley trip value, the rate count is greater than a certain value, and a peak is being sought. As used in FIG. 11, the variable VALUE refers to the measured value of the blood pressure signal at the particular time when algorithm 1100 is performed. In the algorithm illustrated in FIG. 11, a pressure waveform is being tracked. The UP variable determines if a peak (UP=1) or a valley (UP=0) is being sought. The signal transitions when the pressure waveform crosses over the trip value. At this time, the signal will “throw out” the old peak or valley value, and start tracking the current pressure waveform.

If a decision is made in the affirmative at operation 1106, setting operation 1108 performs operations to set UP equal to 1, PEAK equal to VALUE, INIT_CNT equal to INIT_CNT minus one, and RATE_CNT equal to zero. At decision operation 1110 a determination is performed to determine whether the current run of algorithm 1100 is not an initialization run of the algorithm. If it is not, the set valley block 1112 performs an equating operation setting the stored valley value equal to the current valley value.

Decision operation 1114 performs various calculations to determine if the value is less than the peak trip value, a peak is being sought, and the rate count is greater than or equal to a prescribed threshold such as four. If the logical expression is true, then setting operation 1116 assigns the following values to the following variable: UP equal to 0, VALLEY equal to VALUE, INIT_CNT equal to INT_CNT minus one, and RATE_CNT equal to zero. Then, at decision operation 1118, algorithm 1100 determines whether the current run of the algorithm is an initialization run. If not, then the stored peak value is equated with the peak variable at setting operation 1120.

Then, decision operation 1122 determines whether the VALUE is greater than PEAK and whether either a peak is being sought or the current run is an initialization run of the algorithm. If so, the setting operation 1124 sets PEAK equal to the VALUE.

At decision operation 1126, a determination is made whether VALUE is less than valley and whether either a peak is not being sought or the current run is an initialization run of the algorithm. If a positive determination is made then the PEAK is set to the VALUE by setting operation 1128. The flow diagram ends at block 1130.

Exemplary Computer System Overview

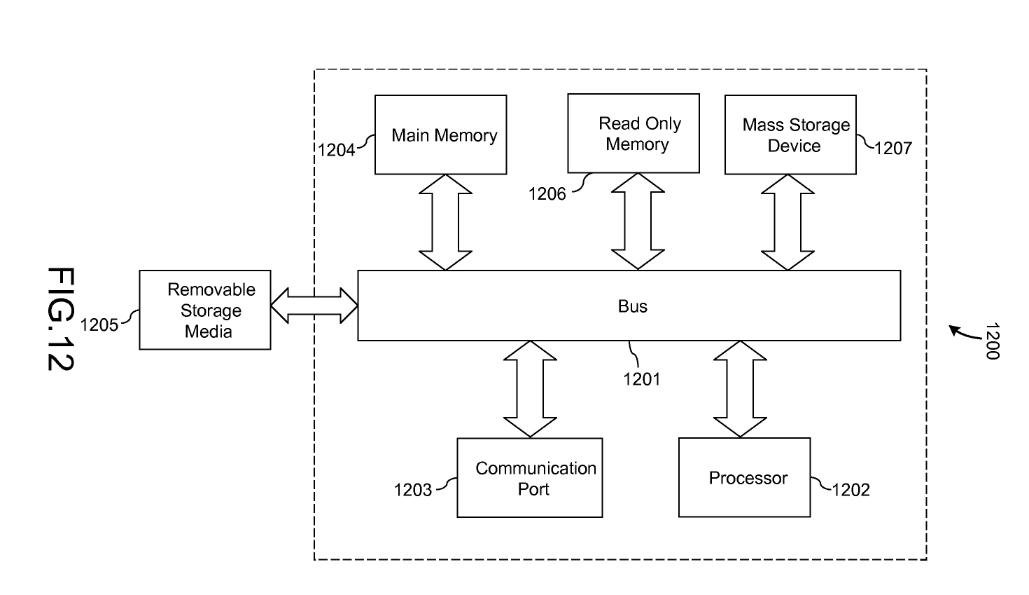

Embodiments of the present invention include various steps a variety of which may be performed by hardware components or may be embodied in machine-executable instructions, which may be used to cause a general-purpose or special-purpose processor programmed with the instructions to perform the steps. Alternatively, the steps may be performed by a combination of hardware, software, and/or firmware. As such, FIG. 12 is an example of a computer system 1200 with which embodiments of the present invention may be utilized. According to the present example, the computer system includes a bus 1201, at least one processor 1202, at least one communication port 1203, and a main memory 1204. System 1200 may also include a removable storage media 1205, a read only memory 1206, and/or a mass storage component/device 1207.

Processor(s) 1202 can be any known processor, including, but not limited to, an Intel® Itanium® or Itanium 2® processor(s), or AMD® Opteron® or Athlon MP® processor(s), or Motorola® lines of processors. Communication port(s) 1203 can be any of an RS-232 port for use with a modem based dialup connection, a 10/100 Ethernet port, or a Gigabit port using copper or fiber. Communication port(s) 1203 may be chosen depending on a network such a Local Area Network (LAN), Wide Area Network (WAN), or any network to which the computer system 1200 connects.

Main memory 1204 can be Random Access Memory (RAM), or any other dynamic storage device(s) commonly known in the art. Read only memory 1206 can be any static storage device(s) such as Programmable Read Only Memory (PROM) chips for storing static information such as instructions for processor 1202.

Mass storage 1207 can be used to store information and instructions. For example, hard disks such as the Adaptec® family of SCSI drives, an optical disc, an array of disks such as RAID, such as the Adaptec family of RAID drives, or any other mass storage devices may be used.

Bus 1201 communicatively couples processor(s) 1202 with the other memory, storage and communication blocks. Bus 1201 can be a PCI/PCI-X or SCSI based system bus depending on the storage devices used.

Removable storage media 1205 can be any kind of external hard-drives, floppy drives, IOMEGA® Zip Drives, Compact Disc—Read Only Memory (CD-ROM), Compact Disc—Re-Writable (CD-RW), Digital Video Disk—Read Only Memory (DVD-ROM).

The components described above are meant to exemplify some types of possibilities. In no way should the aforementioned examples limit the scope of the invention, as they are only exemplary embodiments.

In conclusion, the present invention provides novel systems, methods and arrangements for monitoring physiologic states. While detailed descriptions of one or more embodiments of the invention have been given above, various alternatives, modifications, and equivalents will be apparent to those skilled in the art without varying from the spirit of the invention. Therefore, the above description should not be taken as limiting the scope of the invention, which is defined by the appended claims.

What is claimed is:

1. A method for operating an implantable medical device (IMD), the method comprising:
receiving a physiological signal of a patient;
determining a respiration rate of the patient based on the received physiological signal;
determining with a processor located within the IMD a sampling interval based on the determined respiration rate of the patient;
determining a sampling mode based upon system conditions and external inputs, wherein the sampling mode includes one or more operating parameters; and
recording blood pressure measurements of the patient over the sampling interval according to the one or more operating parameters included in the sampling mode.

2. The method of claim 1, further comprises discriminating between local extrema and global extrema of the blood pressure measurement, wherein discriminating comprises:
setting a discriminator time interval based upon the received physiological signal;
detecting a possible extrema while taking blood pressure measurements during the sampling interval; and
recording the possible extrema if a new extrema is not detected during the discriminator time interval after the detection of the possible extrema.

3. The method of claim 1, wherein the one or more sampling mode parameters are selected from the group consisting of sample rate, maximum sample rate, minimum sample rate, data compression rate, maximum data compression rate, minimum data compression rate, data transfer method, generation of statistical information, sampling interval, maximum sampling interval, minimum sampling interval, choice of physiological signals, use of triggering signal, synchronization mode, accuracy level and measurement frequency.

4. The method of claim 1, wherein the system conditions for determining the sampling mode include remaining power of the IMD and availability of a synchronization signal.

5. The method of claim 1, wherein the external inputs are sent from a second IMD and include one or more of a requested accuracy level, data transfer method, and measurement frequency.

6. The method of claim 1, further comprising:
predicting when an end-expiration ventilation of the patient will occur based on the received physiological signal; and
synchronizing a start of the sampling interval so that the predicted end-expiration ventilation will occur during the sampling interval.

7. An implantable medical device system with reduced power consumption, the system comprising:
a master device configured to receive a first physiological signal of a patient, to determine a sampling interval duration based on the first physiological signal, and to send an instruction containing the sampling interval duration; and
a slave implantable medical device configured to receive the instruction containing the sampling interval duration, to sense a second physiological signal, and to record one or more data points from the second physiological signal over the sampling interval duration.

8. The implantable medical device system of claim 7, wherein the slave implantable medical device is further configured to send the one or more recorded data points to the master device.

9. The implantable medical device system of claim 7, wherein the first physiological signal is a respiration cycle signal, and wherein the second physiological signal is a blood pressure signal.

10. The implantable medical device system of claim 9, wherein the master device is further configured to determine an end expiration point of the respiration cycle signal, wherein the sampling interval duration is smaller than a duration of one full period of the respiration cycle signal, and wherein the instruction further contains synchronization information useable by the slave implantable medical device to coincide the sampling interval duration with the end expiration point.

11. The implantable medical device system of claim 9, wherein the slave implantable medical device is further configured to calculate a statistic of the one or more data points, the statistic selected from the group consisting of: a minimum, a maximum, and an average.

12. The implantable medical device system of claim 11, wherein the master device is further configured to command the slave implantable medical device to send the statistic to the master device.

13. The implantable medical device system of claim 9, wherein the slave implantable medical device is further configured to track peaks and valleys of the blood pressure signal and to record the one or more data points from the blood pressure signal substantially corresponding to peaks or valleys of the blood pressure signal.

14. The implantable medical device system of claim 13, wherein the slave implantable medical device is further configured to discriminate between local extrema and global extrema within the blood pressure signal.

15. The implantable medical device system of claim 9, wherein the respiration cycle signal is a minute ventilation signal.

16. The implantable medical device system of claim 7, wherein the master device is a master implantable medical device.

17. The implantable medical device system of claim 16, wherein the master implantable medical device is a pulse generator.

18. The implantable medical device system of claim 7, wherein the slave implantable medical device comprises:
a battery capable of generating a status signal indicating a current level of charge; and
a sampling mode module to use the status signal to determine an appropriate sampling mode.

19. The implantable medical device system of claim 7, wherein the slave implantable medical device comprises:
a memory to store the one or more data points;
a sampling mode module to determine a sampling mode of a pressure sensor module of the slave implantable medical device, the sampling mode including sample rate, data compression rate, and measurement frequency; and
a communications module to retrieve the one or more data points from the memory and transmit the one or more data points to the master device.

20. The implantable medical device system of claim 9, wherein the instruction includes a timing signal generated by:
determining the duration of the sampling interval so that a desired data point of the blood pressure signal will lie within the sampling interval; and
centering the sampling interval around the desired data point.

21. A method comprising:
monitoring with an implanted sensor module a physiological signal to determine a respiration rate of a patient;
determining with a processor a sampling interval based on the respiration rate of the patient; and
recording blood pressure measurements during the sampling interval.

22. The method of claim 21, further comprising:
predicting when a specific ventilation state will occur based on a received physiological signal; and
synchronizing a start of the sampling interval so that the ventilation state will occur as predicted during the sampling interval.

23. The method of claim 21, further comprising:
receiving a control signal from a master implantable medical device, wherein the control signal includes information about the number of times per day the blood pressure measurements should occur and whether all or a subset of the blood pressure measurements should be transmitted to the master IMD; and
transmitting blood pressure measurements, as indicated by the control signal, to the master IMD.

24. The method of claim 21, further comprising receiving a signal indicating a desired sampling mode, wherein the desired sampling mode includes information about sampling rate, number of measurements per day, and desired compression scheme.
